(19) United States
(12) Patent Application Publication
(10) Pub. No.: US 2008/0119845 A1
(43) Pub. Date: May 22, 2008

(54) FACET REPLACEMENT DEVICE REMOVAL AND REVISION SYSTEMS AND METHODS

(75) Inventors: Martha K. Stone, Lake Forest Park, WA (US); Sean Sung-Ho Suh, Plymouth Meeting, PA (US); Richard J. Broman, Kirkland, WA (US); Anton Alferness, Seattle, WA (US); Anthony V. Finazzo, Lake Forest Park, WA (US); Mark T. Charbonneau, Bellevue, WA (US); Leonard J. Tokish, Issaquah, WA (US)

Correspondence Address: SHAY GLENN LLP, 2755 CAMPUS DRIVE, SUITE 210, SAN MATEO, CA, 94403, US

(73) Assignee: ARCHUS ORTHOPEDICS, INC., Redmond, WA (US)

(21) Appl. No.: 11/861,239

(22) Filed: Sep. 25, 2007

Related U.S. Application Data
(60) Provisional application No. 60/847,013, filed on Sep. 25, 2006.

(51) Int. Cl. A61B 18/18; A61B 17/58; A61H 1/00

(52) U.S. Cl. 606/41; 606/247; 601/2

(57) ABSTRACT

A method and system for removing a portion of an artificial facet from a vertebra, and an adapter within the system that allows ultrasonic energy and extraction forces to be transmitted therethrough are provided. The method includes attaching an adapter to an ultrasonic waveguide and to a stem cemented into a vertebra, and applying ultrasonic energy and extraction force from the waveguide through the adapter to the stem. The system includes a handset that delivers ultrasonic energy, a waveguide attached to the handset to receive the energy therefrom, and an adapter attached to the waveguide to receive the energy therefrom. The adapter includes a first section attaching the adapter to the ultrasonic waveguide, and a second section attaching the adapter to a portion of the artificial facet joint having a stem embedded in a vertebra, the sections of the adapter transmitting energy and forces from the waveguide through the adapter to the attached stem.

### CROSS-REFERENCE TO RELATED APPLICATIONS

This application claims the benefit of U.S. Provisional Application Ser. No. 60/847,013, filed Sep. 25, 2006 and entitled FACET REPLACEMENT DEVICE REMOVAL AND REVISION SYSTEMS AND METHODS.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Back pain, particularly in the small of the back, or lumbosacral region (L4-S1) of the spine, is a common ailment. In many cases, the pain severely limits a person's functional ability and quality of life. Back pain interferes with work, routine daily activities, and recreation. It is estimated that Americans spend $50 billion each year on low back pain alone. It is the most common cause of job-related disability and a leading contributor to missed work.

Through disease or injury, the laminae, spinous process, articular processes, facets and/or facet capsules of one or more vertebral bodies along with one or more intervertebral discs can become damaged, which can result in a loss of proper alignment or loss of proper articulation of the vertebra. This damage can also result in an anatomical change, loss of mobility, and pain or discomfort. For example, the vertebral facet joints can be damaged by traumatic injury or as a result of disease. Diseases damaging the spine and/or facets include osteoarthritis where the cartilage of joints is gradually worn away and the adjacent bone is remodeled, ankylosing spondylolysis (or rheumatoid arthritis) of the spine which can lead to spinal rigidity, and degenerative spondylolisthesis which results in a forward displacement of the lumbar vertebra on the sacrum. Damage to facet joints of the vertebral body often results in pressure on nerves, commonly referred to as “pinched” nerves, or nerve compression or impingement. The result is pain, misaligned anatomy, a change in biomechanics and a corresponding loss of mobility. Pressure on nerves can also occur without facet joint pathology, e.g., as a result of a herniated disc.

One conventional treatment of facet joint pathology is spine stabilization, also known as intervertebral stabilization. Intervertebral stabilization desirably controls, prevents or limits relative motion between the vertebrae through the use of spinal hardware, removal of some or all of the intervertebral disc, fixation of the facet joints, bone graft/osteo-inductive/osteo-conductive material positioned between the vertebral bodies (with or without concurrent insertion of fusion cages), and/or some combination thereof, resulting in the fixation of (or limiting the motion of) any number of adjacent vertebrae to stabilize and prevent/limit/control relative movement between those treated vertebrae.

Although spine fusion surgery is an efficacious treatment, complications can nonetheless result. Patients undergoing spine surgery frequently continue to experience symptoms. For surgical procedures in the lumbar spine, failure rates as high as 37% have been reported after lumbar fusion and 30% for surgery without fusion. See Eichholz, et al., “Complications of Revision Spinal Surgery,” Neurosurg Focus 15(3): 1-4 (2003). Post-operative problems can include decompression related problems, and fusion related problems. Decompression related problems (i.e., loss of normal spine balance resulting in the head and trunk no longer being centered over the pelvis) include, for example, recurrent disc herniation, spinal stenosis, chronic nerve injury, infection, and decompression. Fusion related problems can include, pain from the bone harvest site, failure of a fusion to develop, loosening of the implanted devices, nerve irritation caused by the devices, infection, and poor alignment of the spine.

Stabilization of vertebral bodies can also be achieved (to varying degrees) from a wide variety of procedures, including the insertion of motion limiting devices (such as intervertebral spacers, artificial ligaments and/or dynamic stabilization devices), devices promoting arthrodesis (rod and screw systems, cables, fusion cages, etc.), and complete removal of some or all of a vertebral body from the spinal column (which may be due to extensive bone damage and/or tumorous growth inside the bone) and insertion of a vertebral body replacement (generally anchored into the adjacent upper and lower vertebral bodies). Various devices are known for fixing the spine and/or sacral bone adjacent the vertebra, as well as attaching devices used for fixation.

More recently, various treatments have been proposed and developed as alternatives to spinal fusion. Many of these treatments seek to restore (and/or maintain) some, or all, of the natural motion of the treated spinal unit, and can include intervertebral disc replacement, nucleus replacement, facet joint resurfacing, and facet joint replacement. Such solutions typically include devices that do not substantially impair spinal movement. Thus, spinal arthroplasty has become an acceptable alternative to fusion, particularly in cases of degenerative disc disease. Arthroplasty devices can be particularly useful because the devices are designed to create an artificial joint or restore the functional integrity and power of a joint.

It may be necessary to alter or revise an implanted spinal prosthesis or fusion device. For example, due to the continued progress of spine disease, a spine surgeon may need to remove part or all of a previously implanted arthroplasty device in order to provide access to the patient's vertebra(e) and/or disc. After performing a surgical procedure on the patient (e.g., implantation of an artificial disc, resection of the lamina, etc.), the surgeon may want to provide the patient with a prosthesis to replace the function of the original device or to perform an entirely new function. It some situations, it may be desirable to use a remaining portion of the implanted arthroplasty device as part of the new prosthesis.

A previously implanted arthroplasty device may be anchored into place by a press fit between a portion of the device and a hole formed in the vertebra, a threaded engagement with the bone, and/or a cemented connection. Alternatively or in addition to the above connections, bone growth from the vertebra onto or into the device may be present which creates or strengthens the connection. Accordingly, one or more strong connections between the vertebral bone and portion(s) of the implanted device may need to be broken during a revision surgery. What are needed and are not provided by the prior art are systems, devices and methods allowing a surgeon to easily break the above-described connections without risking damage to the patient's anatomy or the previously implanted device.

### SUMMARY OF THE INVENTION

Embodiments of the invention relate to a method and a system for removing at least a portion of an artificial facet from a vertebra, as well as an adapter within the system that allows ultrasonic energy and extraction forces to be transmitted therethrough.

Embodiments of a method for removing at least a portion of an artificial facet joint from a vertebra include attaching an adapter to an ultrasonic wave guide, attaching the adapter to one of a cephalad stem and a caudal stem cemented into a vertebra, and simultaneously applying ultrasonic energy and an extraction force from a waveguide through the adapter to the stem. The ultrasonic energy being applied may be directed primarily in a torsional direction to the stem, and such energy may further be alternated between at least two different frequencies.

Embodiments of a system for removing at least a portion of an artificial facet joint from a vertebra include a handset configured to deliver ultrasonic energy, a waveguide configured to attach to the handset to receive the ultrasonic energy therefrom, and an adapter configured to attach to the waveguide to receive the ultrasonic energy therefrom, the adapter further being configured to rigidly attach to a portion of an artificial facet joint having a stem embedded in a vertebra in order to be able to transmit ultrasonic energy and extraction forces to the stem. In some of these embodiments the handset may be configured to deliver torsional ultrasonic energy through the waveguide and the adapter to the stem, and in some embodiments, the handset may be configured to deliver ultrasonic energy that alternates between at least two different frequencies.

In some of these system embodiments, the adapter may be configured to rigidly attach to an artificial facet joint portion having an embedded segment and a non-embedded segment, the non-embedded segment being generally perpendicular to the embedded segment, and the adapter being configured to attach to the perpendicular non-embedded segment. In some embodiments, the adapter may include a U-shaped surface configured to receive a bar-shaped section of the perpendicular non-embedded segment of the artificial facet joint portion. In these latter embodiments, the U-shaped surface may have a central axis that forms a non-parallel and non-perpendicular angle with a central axis of the waveguide. In some embodiments, the adapter may further include a movable member for rigidly locking the bar-shaped section against the U-shaped surface. In some embodiments, the adapter may include a U-shaped surface configured with a curved central axis to receive at least a part of a bearing cup of the artificial facet joint portion.

In some embodiments of the system, the adapter includes a feature for receiving the portion of an artificial facet joint, wherein the feature forms a non-orthogonal angle with respect to a central axis of the wave guide so that a central axis of the embedded stem is coplanar with the central axis of the wave guide to increase the extraction force transmitted to the stem. In some of these embodiments, the non-orthogonal angle is about 20 degrees.

Embodiments of the invention further relate to an ultrasonic adapter that includes a first section configured to attach the adapter to an ultrasonic waveguide, and a second section configured to rigidly attach the adapter to a portion of an artificial facet joint having a stem embedded in a vertebra, the first and second sections of the adapter cooperating to allow ultrasonic energy and extraction forces to be transmitted from an attached waveguide through the adapter to an attached stem.

In some embodiments of the ultrasonic adapter, the first section includes a threaded stud that may be receivable in a threaded hole in a waveguide, and a shoulder portion adjacent to the stud that may be configured to abut against a surface adjacent to the threaded hole in the waveguide. In some embodiments of the ultrasonic adapter, the adapter may be configured to deliver torsional ultrasonic energy from a waveguide through the adapter to the stem.

In some embodiments of the ultrasonic adapter configured to deliver torsional energy, the second section may be configured to rigidly attach to an artificial facet joint portion having an embedded segment and a non-embedded segment generally perpendicular to the embedded segment, the second section being configured to attach to the perpendicular non-embedded segment. In some of these embodiments, the second section of the adapter includes a U-shaped surface configured to receive a bar-shaped section of the perpendicular non-embedded segment of the artificial facet joint portion. In various embodiments of the adapter with a U-shaped surface, that surface has a central axis that forms a non-parallel and non-perpendicular angle with a central axis of a waveguide. In some embodiments of the adapter with a U-shaped surface, the second section further includes a movable member for rigidly locking the bar-shaped section against the U-shaped surface.

In some embodiments of the ultrasonic adapter with a first and second section, as summarized above, second section includes a U-shaped surface configured with a curved central axis to receive at least a part of a bearing cup of the artificial facet joint portion. In some embodiments of the summarized ultrasonic adapter, the second section includes a feature for receiving the portion of an artificial facet joint, wherein that feature forms a non-orthogonal angle with respect to a central axis of a wave guide so that a central axis of an embedded stem may be coplanar with the central axis of the wave guide to increase the extraction force transmitted to the stem. In some of these embodiments, the non-orthogonal angle is about 20 degrees.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a posterior prospective view showing two vertebrae having an exemplary implant device attached that may be revised with the inventive devices and methods.

FIG. 2 is a plan view illustrating components of an exemplary embodiment of the inventive ultrasonic revision system.

FIG. 3 is an enlarged view showing a portion of the system of FIG. 2.

FIG. 4 is a perspective view of one embodiment of an adapter tip constructed according to aspects of the present invention.

FIG. 5 is a plan view illustrating components of another exemplary embodiment of the inventive ultrasonic revision system.

FIG. 6 is an enlarged view showing a portion of the system of FIG. 5.

FIG. 7 is a perspective view of another embodiment of an adapter tip constructed according to aspects of the present invention.

FIG. 8 is a side view showing the adapter tip of FIG. 7.

FIG. 9 is an enlarged view showing a portion of the system of FIG. 5.

FIG. 10 is a side view showing a portion of the system of FIG. 5.

FIG. 11 is an enlarged side view showing a portion of the system of FIG. 10.

FIG. 12 is an opposite side view showing a portion of the system of FIG. 5.

FIG. 13 is an enlarged opposite side view showing a portion of the system of FIG. 12.

FIG. 14 is a side view of an adapter wrench constructed according to aspects of the present invention, the wrench being shown in use on an implant embedded in a portion of a spinal column.

### DETAILED DESCRIPTION OF THE INVENTION

The present disclosure relates generally to the field of orthopedic surgery, and more particularly to the instrumentation and techniques for spinal implant revision procedures. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

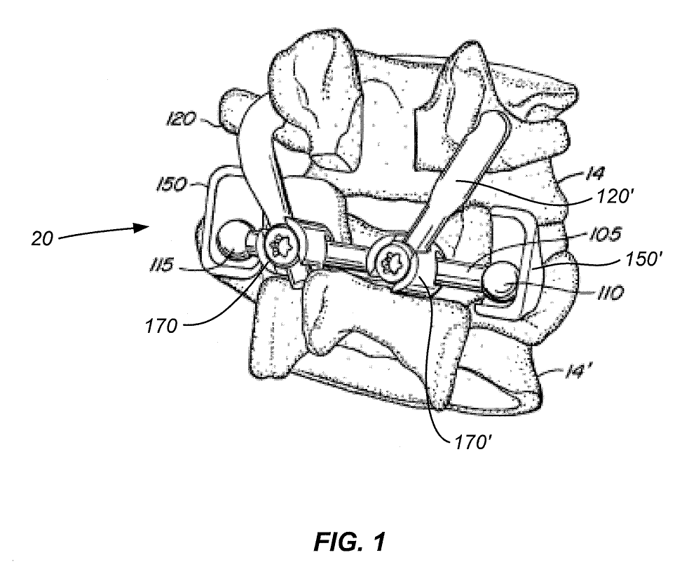

The revision devices and methods of this invention may be used with a variety of spinal implants, such as arthroplasty implants. FIG. I shows an exemplary spinal arthroplasty device 20 attached to adjacent vertebrae 14 and 14′. The spinal arthroplasty device 20 includes a crossbar 105, a pair of cephalad arms 120, 120′ and a pair of caudal cups 150, 150′. Heads 110 and 115 at opposing ends of crossbar 105 interact with bearing surfaces inside caudal cups 150 and 150′ to replace the articulating action of the patient's natural facet joints, which have been removed, when the patient flexes and extends his or her back. In this example, each cephalad arm 120, 120′ attaches to the pedicle of the superior vertebra 14 as shown, via, e.g., a stem (not seen in FIG. 1) inserted into the pedicle. The other ends of the cephalad arms attach to crossbar 105 via crossbar mounts 175 and 175′. The caudal cups 150 and 150′ attach to the inferior vertebra 14′ via, e.g., stems (not seen in FIG. 1) inserted into the pedicles. Further details of this exemplary spinal arthroplasty device 20 may be found in U.S. Ser. No. 11/206,676.

The exterior surfaces of the cephalad and caudal stems may include textures or coatings which enhance the fixation of the implanted prosthesis by promoting bone growth in and around the implanted prosthesis. For example, the surfaces may be roughened such as by chemical etching, bead-blasting, plasma spray porous coating, sanding, grinding, serrating, and/or diamond-cutting. All or a portion of the exterior surfaces may also or alternatively be coated with biocompatible osteoconductive materials such as hydroxyapatite (HA) or osteoinductive coatings such as bone-morphogenic proteins. Permanent or temporary adhesive materials may also be used to hold the implant 20 in place until bone growth has advanced to provide more stable fixation. In some embodiments, PMMA bone cement is the preferred adhesive material. An heat insulating material may also be used in the bone adjacent to the implant. The cement itself, or additives to the cement, can provide the heat insulation. This insulation allows the cement to be later disrupted in a revision procedure, but insulates the surrounding bone from the heating side effects of the disruption.

After the implant 20 becomes affixed, conditions may arise, such as additional spinal disease or injury, deterioration of the implant, migration of the implant, or improvements in technology, which require revision of the implant. In the above exemplary embodiment, such revision surgery may require accessing implant 20 from a posterior approach, removing or repositioning one or both cephalad stems from vertebra 14, and/or removing or repositioning one or both caudal stems from vertebra 14′. Before removing or repositioning one or more stems, implant 20 may be disassembled by loosening and removing crossbar mounts 170, 170′, crossbar 105, and/or heads 110 and 115. Cephalad arms 120, 120′ may also first be removed if they are not integral with the cephalad stems. Similarly, caudal cups 150, 150′ may also be removed if they are not integral with the caudal stems.

The application of heat is one technique that may be used to weaken and soften the cement securing stems 180, 180′, 190, 190′ in their respective vertebrae 14, 14′. However, the direct application of heat to the stems (rather than using ultrasonic energy) often results in significant amounts of waste heat that can go into the surrounding tissue, such as the softer tissues adjacent to the implant, prior to the implant heating up enough to soften the surrounding cement. The application of ultrasonic energy, in contrast to direct heat, allows for heating of the cement (thereby weakening the cement) by vibrating the implant, which then heats the cement through direct contact between the cement and implant as well as vibratory friction between the implant and the cement. The movement simultaneously shakes the mechanical bonds in the weakened cement. The application of both heat and vibration to the cement at the same time (provided by the ultrasonic energy) provides faster breakdown of the cement without causing unacceptable damage to surrounding healthy tissues.

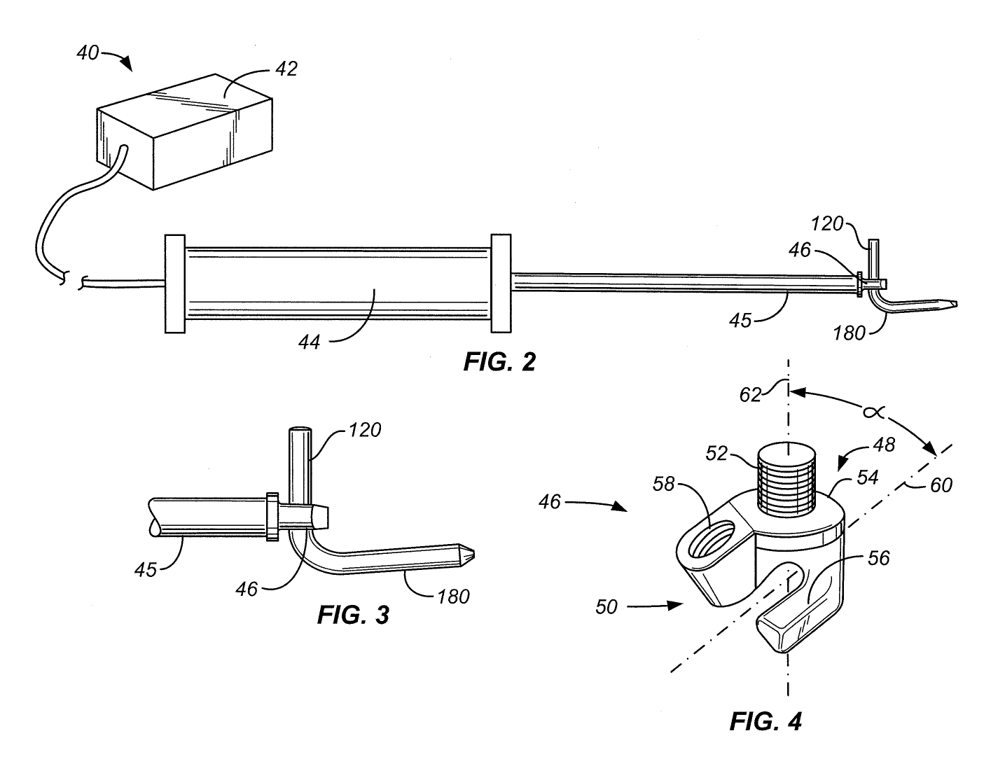

Referring to FIG. 2, an ultrasonic revision tool 40 may be used to loosen and/or remove the spinal implant 20. The tool 40 may be used in any area of the spine including the cervical area. The tool 40 may also be used with other types of spinal implants, such as dynamic stabilizers, fusion systems, other types of facet joint replacements, and disc replacements. The tool 40 may include a power supply device 42, a handset 44, a waveguide 45 and an ultrasonic adapter tip 46 configured to rigidly attach to cephalad arm 120. In this exemplary embodiment, cephalad arm 120 of implant 20 is shown integrally formed with cephalad stem 180, best seen enlarged in FIG. 3. Cephalad arm 120 and cephalad stem 180 may be formed by bending a single bar of implantable material such that arm 120 and stem 180 are generally perpendicular to each another.

The power supply device 42 may include a variety of devices (not shown) including an ultrasonic generator, frequency adjustment controls, fluid delivery controls, and other devices which may allow the operator to control the ultrasonic revision tool 40. The handset 44 may include an actuator (not shown), such as a transducer, for converting electrical ultrasonic energy into mechanical ultrasonic vibratory motion having a frequency in the ultrasonic range, i.e. greater than 20 kilohertz.

Referring to FIG. 4, the cephalad adapter tip 46 may include a first section 48 for attaching the adapter 46 to waveguide 45, and a second section 50 for attaching the adapter 46 to a portion of implant 20, such as cephalad arm 120. In this exemplary embodiment, first section 48, comprises a threaded stud 52 which is received in a mating threaded hole in the distal end of waveguide 45. A wrench or other tool may be used to tighten adapter 46 into the end of waveguide 45, such that shoulder surface 54 is pressed against a mating surface on the distal end of waveguide 45. The first section 48 may be provided with other features instead of stud 52 and shoulder 54 that allow adapter 46 to be removably attached to waveguide 45.

The second section of adapter 46 may be provided with a V-shaped or U-shaped surface 56 for removably receiving cephalad arm 120 transversly therethrough. A threaded hole 58 may also be provided to secure arm 120 against surface 56, such as by tightening a set screw in hole 58 against arm 120 so that it is urged against the opposite side of U-shaped surface 56. The set screw may be provided with buttress threads which resist screw loosening. Other movable members or locking features may be used instead of a set screw in hole 58. For example, a threaded knob or lever may be used with hole 58. Alternatively, a twisting cam lock may be used to secure arm 120 against surface 56.

As shown in FIG. 4, U-shaped surface 56 may be oriented such that its central axis 60 forms an angle a with a central axis 62 of stud 52 and waveguide 45. In some embodiments, angle α is between 0 and 90 degrees, such as 45 degrees, to aid the surgeon in engaging cephalad arm 120 while it is still implanted, and to provide the surgeon with adequate access to a set screw in threaded hole 58.

In some embodiments it may be desirable to orient the locking screw or other device along the longitudinal axis of vibratory motion. This avoids a situation where a locking device locks transverse to the ultrasonic vibration and may allow the vibrating tip to slide along the implant without transferring a significant amount of energy. Properly locking the ultrasonic adapter to the implant allows sufficient transfer of energy from the waveguide to the implant. The implant in turn is then able to transfer the energy to the surrounding tissues or materials needing disruption. The relatively small mass of most spinal implants lends itself to such vibration.

The components of the revision tool 40 may be made of durable, medically acceptable materials, such as stainless steel, hard coated anodized aluminum, or titanium, for example, capable of being sterilized to medical standards, such as by steam or flash autoclaving, gas sterilization, and/or soaking in a disinfectant solution. Accordingly, the revision tool 40 may or may not be designed for repeated use. In alternative embodiments, to promote efficiency and sterility, ultrasonic adapter tip 46 may be disposable.

In operation, the power supply device 42 may provide a high frequency, low amplitude ultrasonic energy to handset 44 which may, in turn, supply ultrasonic vibratory motion through waveguide 45 to the ultrasonic adapter 46. In some embodiments, the ultrasonic frequency may be 20 kilohertz or greater. The ultrasonic motion may be directed in an axial, radial or torsional direction, or a combination thereof. While adapter 46 is moving with ultrasonic frequency, its actual displacement may be relatively small, for example less than a few millimeters. A tight connection between the shoulder surface 54 of adapter 46 and the distal end of waveguide 45 may serve to efficiently transfer ultrasonic vibratory motion from the revision tool 40 to the implant 20. The ultrasonic vibratory motion transferred from adapter 46 to cephalad arm 120 and stem 180 may fragment the adjacent bone ingrowth and overgrowth, causing stem 180 to break loose from vertebral body 14 with minimal trauma to surrounding bone or soft tissue. Because stem 180 may be held in place by a combination of mechanical features (e.g., surface textures, tabs, anchors) and bone overgrowth and ingrowth, the ultrasonic motion may act upon the bone adjacent to the implant rather than on a cement mantle. In other embodiments, the ultrasonic frequency may be selected such that the applied energy is focused on the cement. In these embodiments, the cement may be fractured and/or melted while the surrounding bone is relatively unaffected. This may be particularly advantageous when it is desirable to remove an implanted stem from a vertebra pedicle. In this situation, the stem may be removed from a small diameter pedicle without requiring removal of the entire cement bolus. Otherwise, the cement mantle might make the implant too large to pull out of the pedicle without removing most of the bone in that area (e.g. the entire pedicle.). By focusing the ultrasonic energy on the cement rather than the bone, the cement can be softened or removed and the implant simply pulled free from the intact pedicle.

As stem 180 begins to loosen, ultrasonic vibratory motion may be supplemented with larger movements of the handset 44, waveguide 45 and adapter 46 such as arc shaped motions, linear reciprocating motions, or random motions to further loosen stem 180. The speed, force, and other characteristics of the movement of adapter 46 may be adjusted, for example, by varying the ultrasonic frequency or amplitude. The displacement of adapter 46 and stem 180 caused by the ultrasonic vibratory motion may be less than a few millimeters, however larger or smaller displacements may be appropriate for certain applications. An optimized frequency or frequencies for each particular revision component to be removed or repositioned can be determined with computer modeling and/or empirically by varying frequency over a range and monitoring the resulting movement, temperature or results. In some embodiments, system 40 may be configured to alternate between two or more frequencies. This may be advantageous when one frequency is found to be optimal for vibrating a particular bone cement and a different frequency is found to be optimal for vibrating a particular portion of the implant. In some embodiments, a user may also be permitted to adjust the duty cycle (i.e. the percentage of time) of each of the alternating frequencies.

The revision tool 40 may eliminate or reduce the need for sharp chisels, hammers, or other instruments which can potentially damage surrounding tissue and/or severely injure the patient. As described, the tool 40 may break up hard tissue in the area of application, but may have a relatively benign impact on surrounding soft tissue. Bone ingrowth and overgrowth can obscure the size and shape of the implant. In this environment, the disclosed tool 40 may minimize the damage caused to the vertebral bodies by targeting the bone removal to the areas most proximate to the implant without requiring a clear view of the implant. The use of ultrasonic vibration to remove or relocate an implant may also promote the long term stability of a repositioned or replacement implant because the bone particles released by the vibration may be redeposited in the area of the implant to stimulate subsequent bone ingrowth around a subsequent implant.

In some embodiments of an ultrasonic revision system and technique, an ultrasonic tool tip may be located at or near the margin of the bone/ implant interface rather than or in addition to coupling directly to the implant. The tool tips may be straight, curved, hooked or other shape to provide versatility in accessing and applying ultrasonic energy to the bone/implant interface. The engagement portions may be osteotomes, files, or other types of sculpting and separating instruments. With such arrangements, ultrasonic vibratory motion may be passed through the tool tip causing the bone and/or cement surrounding the implant to crumble or break loose. Once the implant or portion thereof is removed from the patient, ultrasonic tool tips may also be used to separate and remove unwanted bony deposits that have developed on the implant.

Once an implant or a portion thereof is loosened, it may be removed from the bone. The vacated bone channel can then be redrilled or used as is for implantation of a repaired or different device. In some procedures, however, removal of the implant or portion may not be desired. Ultrasonic system 40 may be used to loosen or melt cement securing an implant in a bone. The implant may then be repositioned without removing it. The original cement may then be allowed to reharden, securing the implant in the new position. In some procedures, the original cement may be augmented or displaced with fresh cement.

In some embodiments, the ultrasonic revision tool 40, may further include a fluid delivery system that applies a fluid such as water to the area of the engagement portion as the implant is loosened. This lavage may act as a coolant, a lubricant, and/or a cleansing agent.

Although an electrically powered revision instrument has been disclosed above, it is understood that alternative power devices may be selected including pneumatic, battery, or gas powered devices. These alternative power devices may be supported by additional or alternative components. Also the components of the power supply device may be integrally formed with the handset.

In some embodiments, the ultrasonic system 40 may be configured to apply heat directly to the implant, in combination with delivering ultrasonic energy. A heating element, much like that of a soldering gun, may be located in handset 44, waveguide 45 and/or adaptor 46, with the components being configured for proper heat conduction to the implant. The direct heat may be applied before the ultrasonic energy to pre-heat the implant. Alternately, or in combination, the direct heat may be applied during or after application of ultrasonic energy.

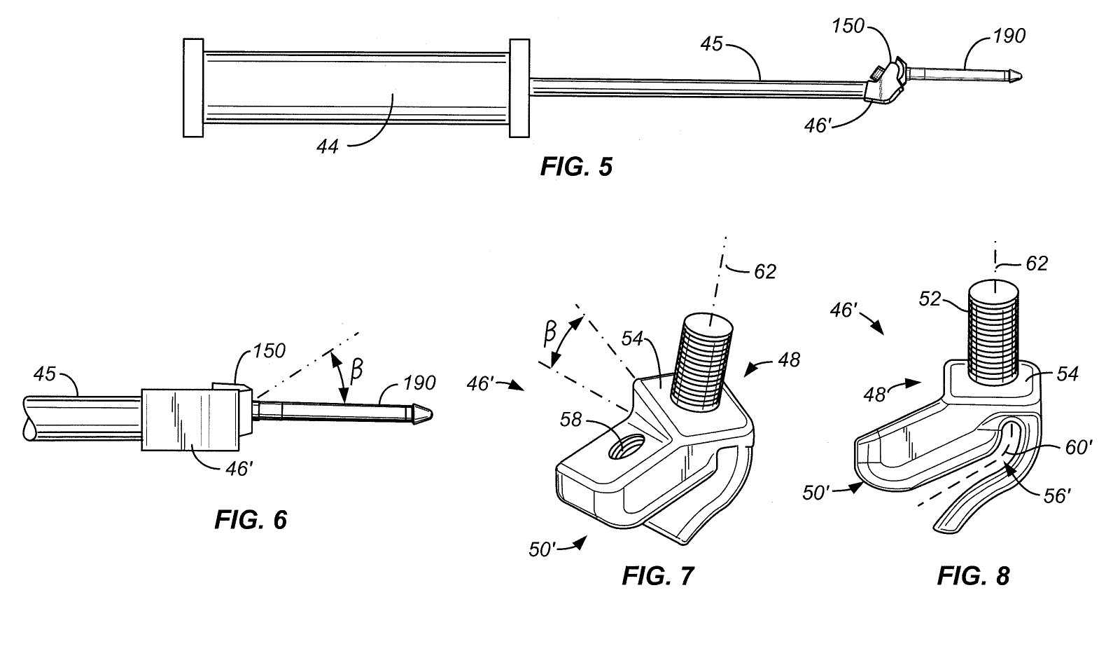

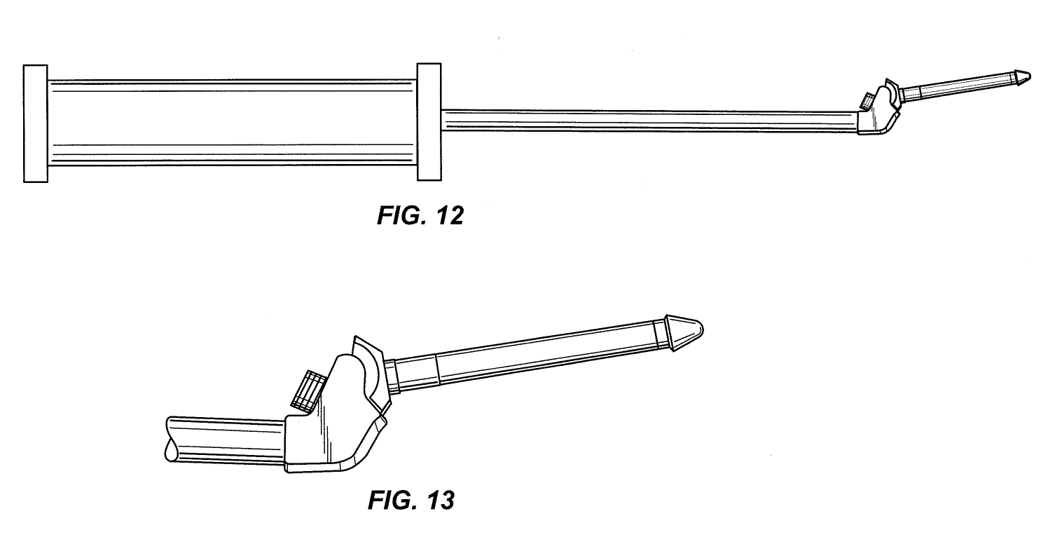

Referring now to FIGS. 5-13, an ultrasonic system 40′ is described for removing or repositioning the caudal stems 190, 190′ of implant 20. System 40′, part of which is shown in FIGS. 5 and 6, is similar to system 40 described above for the removal or repositioning of cephalad stems 180, 180′, but system 40′ is fitted with a different adapter 46′ configured specifically for removably attaching to caudal bearing cups 150, 150′ which are connected to caudal stems 190, 190′, respectively.

Referring to FIGS. 7 and 8, the caudal adapter tip 46′ may include a first section 48 for attaching the adapter 46′ to waveguide 45, and a second section 50′ for attaching the adapter 46′ to a different portion of implant 20, such as a caudal bearing cup 150 connected to a caudal stem 190. As with adapter 46 described above, first section 48 comprises a threaded stud 52 which is received in a mating threaded hole in the distal end of waveguide 45. A wrench or other tool may be used to tighten adapter 46′ into the end of waveguide 45, such that shoulder surface 54 is pressed against a mating surface on the distal end of waveguide 45. The first section 48 may be provided with other features instead of stud 52 and shoulder 54 that allow adapter 46 to be removably attached to waveguide 45.

The second section of adapter 46′ may be provided with a U-shaped surface or groove 56′ for removably receiving a portion of a caudal bearing cup 150. In some embodiments, groove 56′ fits inside the bearing surface of caudal bearing cup 150 and around the superior and medial edges of the cup. As can been seen in FIG. 8, groove 56′ has a centerline 60′ that is curved to match the corresponding contour of cup 150. A threaded hole 58 may also be provided to secure cup 150 in groove 56′, such as by tightening a set screw in hole 58 against cup 150 so that it is urged against the opposite side of U-shaped surface 56′. In some embodiments, the set screw is tightened enough for its tip to penetrate the surface of cup 150, to ensure maximum transmission of ultrasonic energy from adapter 46′ to cup 150. Other movable members or locking features may be used instead of a set screw in hole 58. For example, a threaded knob or lever may be used with hole 58. Alternatively, a twisting cam lock may be used to secure cup 150 against surface 56′.

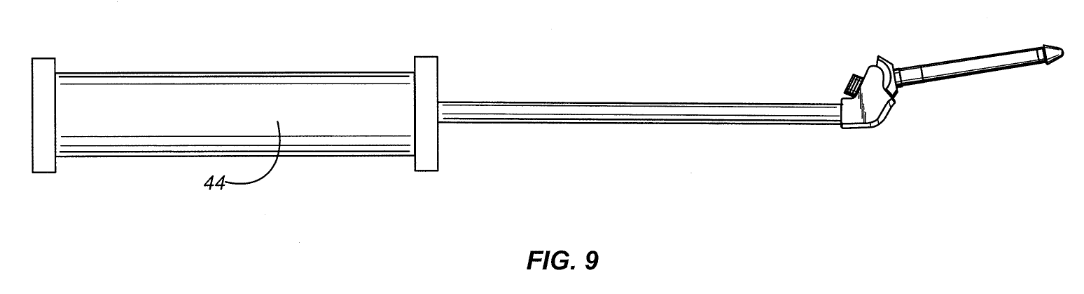

As best seen in FIG. 7, U-shaped surface 56 and threaded hole 58 may be set at an angle β with respect to a plane orthogonal to a central axis 62 of mounting stud 52 and waveguide 45. This angle may be configured to match an angle of tilt P between caudal cup 150 and stem 190, shown in FIG. 6. With this arrangement, a central axis of caudal stem 190 is more closely aligned (i.e. is parallel or at least coplanar) with central axis 62 of waveguide 45, as shown in FIGS. 6 and 9. In this manner, a greater percentage of a surgeon's pulling force acts to remove stem 190. In the exemplary embodiment shown, angle β is about 20 degrees.

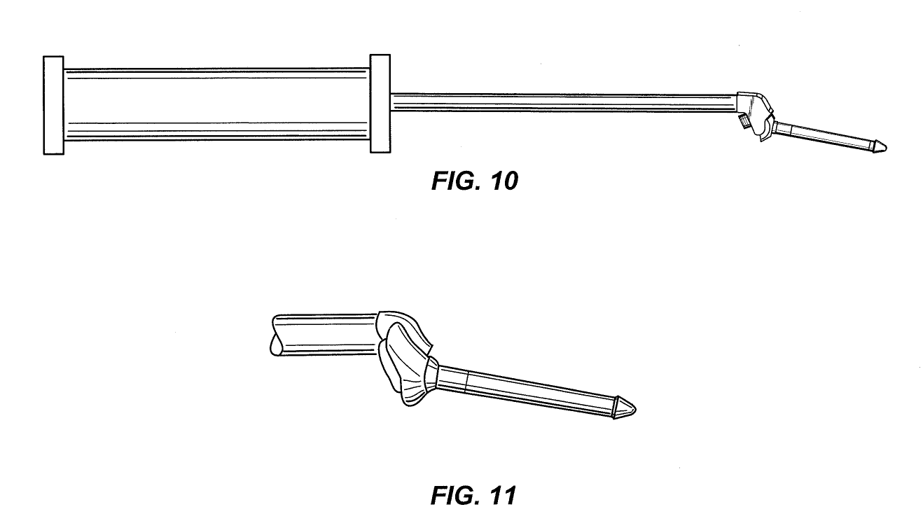

Referring to FIGS. 10-13, additional side views of ultrasonic system 40′ are shown. Among other aspects, these figures illustrate that adapter 46′ can be configured to align a central axis of caudal stem 190 in a non-parallel manner with a central axis of waveguide 45, but that the two axes are still coplanar (as shown in FIGS. 6 and 9).

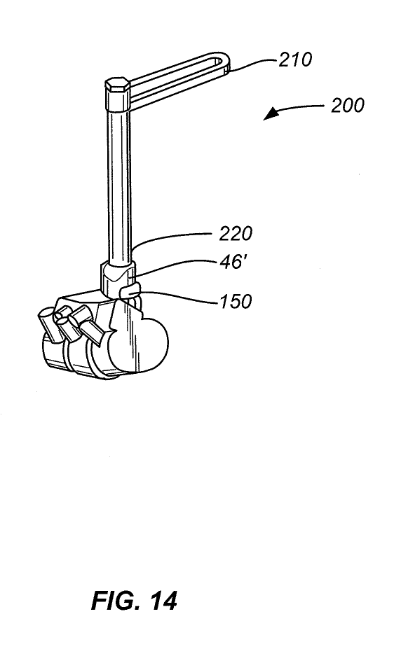

Referring to FIG. 14, an adapter wrench 200 constructed according to aspects of the present invention is shown. In this exemplary embodiment, wrench 200 comprises a handle 210 extending laterally from its proximal end. The distal end 220 of wrench 200 is provided with an attachment feature (not shown), such as a threaded hole, similar to the hole in the distal end of waveguide 45 described above, for receiving a mating feature, such as threaded stud 52 on adapter 46′, shown in FIG. 7 and described above. As shown in FIG. 14, adapter 46′ may be attached to the distal end 220 of wrench 200 using the attachment feature. Adapter 46′ may then be attached to caudal bearing cup 150, and handle 210 may be used to apply torque and/or other loosening or extraction forces to the implant to facilitate its repositioning or removal.

Wrench 200 may be used after or between applications of ultrasonic energy applied by system 46′ to assist in removing or repositioning the implant. Various adapter tips, such as 46 and 46′ described above, may be alternately attached to wrench 200 for use on particular implant components. In other embodiments, each adapter may be integrally formed on its own wrench, and a set of such wrenches provided to a surgeon performing revision surgery.

Although several exemplary embodiments of this invention have been described in detail above, those skilled in the art will readily appreciate that many modifications are possible in the exemplary embodiments without materially departing from the novel teachings and advantages of this invention. Accordingly, all such modifications and alternative are intended to be included within the scope of this invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure.

What is claimed is:

1. A method of removing or repositioning at least a portion of an artificial facet joint with respect to a vertebra, the method comprising:
attaching an adapter to an ultrasonic wave guide;
attaching the adapter to one of a cephalad stem and a caudal stem cemented into a vertebra; and
simultaneously applying ultrasonic energy and an moving force from the waveguide through the adapter to the stem.

2. The method of claim 1, wherein the ultrasonic energy is directed primarily in a torsional direction to the stem.

3. The method of claim 1, wherein the applied ultrasonic energy is alternated between at least two different frequencies.

4. A system for removing or repositioning at least a portion of an artificial facet joint with respect to a vertebra, the system comprising:
a handset configured to deliver ultrasonic energy;
a waveguide configured to attach to the handset to receive the ultrasonic energy therefrom; and
an adapter configured to attach to the waveguide to receive the ultrasonic energy therefrom, the adapter further configured to rigidly attach to a portion of an artificial facet joint having a stem embedded in a vertebra to transmit ultrasonic energy and moving forces to the stem.

5. The system of claim 4, wherein the handset is configured to deliver torsional ultrasonic energy through the waveguide and the adapter to the stem.

6. The system of claim 4, wherein the handset is configured to deliver ultrasonic energy that alternates between at least two different frequencies.

7. The system of claim 4, wherein the adapter is configured to rigidly attach to an artificial facet joint portion having an embedded segment and a non-embedded segment generally perpendicular to the embedded segment, the adapter being configured to attach to the perpendicular non-embedded segment.

8. The system of claim 7, wherein the adapter comprises a U-shaped surface configured to receive a bar-shaped section of the perpendicular non-embedded segment of the artificial facet joint portion.

9. The system of claim 8, wherein the U-shaped surface has a central axis that forms a non-parallel and non-perpendicular angle with a central axis of the waveguide.

10. The system of claim 8, wherein the adapter further comprises a movable member for rigidly locking the bar-shaped section against the U-shaped surface.

11. The system of claim 4, wherein the adapter comprises a U-shaped surface configured with a curved central axis to receive at least a part of a bearing cup of the artificial facet joint portion.

12. The system of claim 4, wherein the adapter comprises a feature for receiving the portion of an artificial facet joint, wherein the feature forms a non-orthogonal angle with respect to a central axis of the wave guide so that a central axis of the embedded stem is coplanar with the central axis of the wave guide to increase the moving force transmitted to the stem.

13. The system of claim 12, wherein the non-orthogonal angle is about 20 degrees.

14. An ultrasonic adapter comprising:
a first section configured to attach the adapter to an ultrasonic waveguide; and
a second section configured to rigidly attach the adapter to a portion of an artificial facet joint having a stem embedded in a vertebra, the first and second sections of the adapter cooperating to allow ultrasonic energy and moving forces to be transmitted from an attached waveguide through the adapter to an attached stem.

15. The ultrasonic adapter of claim 14, wherein the first section comprises a threaded stud receivable in a threaded hole in a waveguide, and a shoulder portion adjacent to the stud configured to abut against a surface adjacent to the threaded hole in the waveguide.

16. The ultrasonic adapter of claim 14, wherein the adapter is configured to deliver torsional ultrasonic energy from a waveguide through the adapter to the stem.

17. The ultrasonic adapter of claim 16, wherein the second section is configured to rigidly attach to an artificial facet joint portion having an embedded segment and a non-embedded segment generally perpendicular to the embedded segment, the second section being configured to attach to the perpendicular non-embedded segment.

18. The ultrasonic adapter of claim 17, wherein the second section comprises a U-shaped surface configured to receive a bar-shaped section of the perpendicular non-embedded segment of the artificial facet joint portion.

19. The ultrasonic adapter of claim 18, wherein the U-shaped surface has a central axis that forms a non-parallel and non-perpendicular angle with a central axis of a waveguide.

20. The ultrasonic adapter of claim 18, wherein the second section further comprises a movable member for rigidly locking the bar-shaped section against the U-shaped surface.

21. The ultrasonic adapter of claim 14, wherein the second section comprises a U-shaped surface configured with a curved central axis to receive at least a part of a bearing cup of the artificial facet joint portion.

22. The ultrasonic adapter of claim 14, wherein the second section comprises a feature for receiving the portion of an artificial facet joint, wherein the feature forms a non-orthogonal angle with respect to a central axis of a wave guide so that a central axis of an embedded stem is coplanar with the central axis of the wave guide to increase an extraction force transmitted to the stem.

23. The ultrasonic adapter of claim 22, wherein the non-orthogonal angle is about 20 degrees.
